(19) United States
(12) Patent Application Publication
(10) Pub. No.: US 2025/0242348 A1
(43) Pub. Date: Jul. 31, 2025

(54) NANOPORE SEQUENCING SYSTEMS

(71) Applicant: ILLUMINA, INC., San Diego, CA (US)

(72) Inventors: Xu Liu, Foster City, CA (US); John Walker, Richardson, TX (US)

(21) Appl. No.: 18/852,418

(22) PCT Filed: Aug. 29, 2023
(86) PCT No.: PCT/US2023/073070

§ 371 Date: Sep. 27, 2024

Related U.S. Application Data
(60) Provisional application No. 63/379,431, filed on Oct. 13, 2022.

(51) Int. Cl. B01L 3/00; C12Q 1/6869; G01N 33/487

(52) U.S. Cl. CPC B01L 3/502761; C12Q 1/6869; G01N 33/48721; B01L 2300/0645; B01L 2300/0663; B01L 2300/0819; B01L 2300/0893; B01L 2300/0896

(57) ABSTRACT

In one aspect, the disclosed technology relates to systems and methods for compensating offset voltages in electrodes when using nanopore sensors to sequence polynucleotides. In one embodiment, the disclosed system for sequencing polynucleotides is fluidically connected to a cis electrode and a trans electrode and the disclosed system includes: a nanopore for sensing a polynucleotide; an amplifier configured to measure an electrical response associated with the nanopore; and a bias compensation circuit coupled to the nanopore and the amplifier, the bias compensation circuit configured to: (i) store a voltage potential indicative of a first offset voltage in the cis electrode and a second offset voltage in the trans electrode, and (ii) compensate the first offset voltage and the second offset voltage using the voltage potential.

### CROSS-REFERENCE TO RELATED APPLICATIONS

This application claims priority to U.S. Provisional Application No. 63/379,431, filed Oct. 13, 2022, the content of which is incorporated by reference in its entirety.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic electrical current. These characteristic electrical currents of the traversing polynucleotide can be recorded to determine the sequence of the polynucleotide.

### SUMMARY

Provided in examples herein are methods for sequencing biopolymers, particularly polynucleotides, and systems and kits for performing the methods.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

During use of nanopore sequencing systems, the concentration of electrolytes in the cis well/chamber and the trans well/chamber changes, and in some cases the electrode(s) are also consumed, due to electrochemical processes (which may be faradaic processes or non-faradaic processes), which result in an extra voltage potential described by the Nernst equation. This extra voltage potential affects the accuracy of the voltage bias that is intended to be applied across the nanopore. This extra voltage potential or “offset potential” varies at different times during use of the nanopore sequencing systems. Moreover, in a nanopore sequencing system comprising an array of nanopores and cis/trans wells/chambers, each pixel (i.e., each nanopore sequencing unit cell) may have a different concentration of electrolytes and may have a different consumption rate of the electrolytes and/or electrodes. As such, voltages across the nanopores may not be accurately measured to identify the polynucleotides that are passing the nanopores. To address the above problems, in certain embodiments, the nanopore system includes an offset capacitor to reduce the pixel-to-pixel variation and/or the time variation due the differing electrolyte concentration and electrolyte/electrode(s) consumption of each nanopore sequencing unit cell.

In some embodiments, the disclosed technology relates to using offset capacitor(s) in the electrical circuit of a pixel. For example, the circuit may be designed such that the offset potential is stored in a capacitor by closing all the “reset” switches while keeping the “sample” switch open. During the measurement phase, one may open all the “reset” switches and close the “sample” switch to measure electrical responses in the pixel. The offset potential resulting from the consumption of electrodes and electrode and/or changed in electrolyte concentration may be canceled out by the voltage stored in the offset capacitor.

In addition, the amplifier used to measure electrical responses in the pixel may have an input offset voltage, and such input offset voltage can be canceled in a similar way (i.e., the way that the offset potential associated with the electrodes is cancelled) by using another capacitor to store the input offset voltage of the amplifier before the measurement phase.

In some embodiments, disclosed is an analog mechanism and a circuit design that can be used to compensate the variation of the voltage bias on a nanopore/amplifier array. In some embodiments, the disclosed technology comprises using an offset capacitor to measure and store the offset voltage of each pixel, wherein the offset voltage would cancel out any input offset voltage in the amplifier array.

Additional details of exemplary nanopore sequencing devices and methods of operating the devices that can be used in conjunction with the present disclosure can be found in U.S. Provisional Patent Application Nos. 63/200,868 and 63/169,041 (International Patent Application Numbers PCT/US2021/038125 and PCT/US2022/020395) and PCT/US2022/049414 and PCT/US2023/013810, the entirety of each of the disclosures is incorporated herein by reference.

In some embodiments, the techniques described herein relate to a device for sequencing polynucleotides including at least one sequencing cell, wherein the at least one sequencing cell includes: a nanopore disposed between a cis electrode and a trans electrode for sensing a polynucleotide; an amplifier configured to measure an electrical response associated with the nanopore; and a bias compensation circuit coupled to the nanopore and the amplifier, the bias compensation circuit configured to: store a first voltage potential indicative of a first offset voltage in the cis electrode and a second offset voltage in the trans electrode; and compensate the first offset voltage and the second offset voltage using the first voltage potential.

In some embodiments, the techniques described herein relate to a device, wherein the bias compensation circuit includes a first offset capacitor, and wherein the first offset capacitor stores the first voltage potential indicative of the first offset voltage in the cis electrode and the second offset voltage in the trans electrode during a first operation mode.

In some embodiments, the techniques described herein relate to a device, wherein the first offset capacitor has a first terminal and a second terminal, and wherein during the first operation mode: the first terminal is connected to the trans electrode; and the second terminal is operably connected to the cis electrode.

In some embodiments, the techniques described herein relate to a device, wherein the first offset capacitor compensates the first offset voltage and the second offset voltage using the first voltage potential during a second operation mode, and wherein the amplifier measures the electrical response associated with the nanopore during the second operation mode.

In some embodiments, the techniques described herein relate to a device, wherein the amplifier has a bias offset voltage between a first input terminal and a second input terminal, and wherein the bias compensation circuit includes a second offset capacitor that stores a second voltage potential indicative of the bias offset voltage during the first operation mode.

In some embodiments, the techniques described herein relate to a device, wherein the second offset capacitor has a third terminal and a fourth terminal, and wherein during the first operation mode: the third terminal is operably connected to the second input terminal; and the fourth terminal is connected to the first input terminal.

In some embodiments, the techniques described herein relate to a device, wherein the second offset capacitor compensates the bias offset voltage using the second voltage potential during the second operation mode.

In some embodiments, the techniques described herein relate to a device, wherein the first offset voltage results from a first redox reaction in the cis electrode and the second offset voltage results from a second redox reaction in the trans electrode.

In some embodiments, the techniques described herein relate to a device, wherein the first offset voltage and the second offset voltage are time-varying.

In some embodiments, the techniques described herein relate to a device, wherein the nanopore is a polypeptide nanopore or a solid-state nanopore, and wherein the polypeptide nanopore forms an opening in a lipid, polymer, or solid-state membrane.

In some embodiments, the techniques described herein relate to a device, wherein the electrical response is an ionic current through the nanopore, and wherein the ionic current is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

In some embodiments, the techniques described herein relate to a method for sequencing polynucleotides, including: providing a polynucleotide to a sequencing cell including a nanopore disposed between a cis electrode and a trans electrode, an amplifier for measuring an electrical response associated with the nanopore, and a bias compensation circuit between the nanopore and the amplifier; storing, by the bias compensation circuit, a first voltage potential indicative of a first offset voltage in the cis electrode and a second offset voltage in the trans electrode; and measuring the electrical response, wherein the first offset voltage and the second offset voltage are compensated using the first voltage potential.

In some embodiments, the techniques described herein relate to a method, wherein the first voltage potential is stored in a first offset capacitor of the bias compensation circuit during a first operation mode.

In some embodiments, the techniques described herein relate to a method, wherein storing the first voltage potential includes: connecting a first terminal of the first offset capacitor to the trans electrode; and connecting a second terminal of the first offset capacitor to the cis electrode.

In some embodiments, the techniques described herein relate to a method, wherein measuring the electrical response includes: connecting the first terminal of the first offset capacitor to the trans electrode; and connecting the second terminal of the first offset capacitor to a first input terminal of the amplifier.

In some embodiments, the techniques described herein relate to a method, wherein the amplifier has a bias offset voltage between the first input terminal and a second input terminal, the method further including: storing, by the bias compensation circuit, a second voltage potential indicative of the bias offset voltage during the first operation mode.

In some embodiments, the techniques described herein relate to a method, wherein measuring the electrical response further includes compensating the bias offset voltage using the second voltage potential during a second operation mode.

In some embodiments, the techniques described herein relate to a method, wherein the electrical response measured by the amplifier is an ionic current through the nanopore or equivalents thereof.

In some embodiments, the techniques described herein relate to a method, further including providing an electrolyte to the sequencing cell prior to providing the polynucleotide.

In some embodiments, the techniques described herein relate to a system for sequencing polynucleotides, including: a plurality of sequencing cells, each of the plurality of sequencing cells including: a nanopore disposed between a cis electrode and a trans electrode for sensing a polynucleotide; an amplifier configured to measure an electrical response in the nanopore; and a bias compensation circuit configured to: store a voltage potential indicative of a first offset voltage in a common cis electrode and a second offset voltage in the trans electrode; and compensate the first offset voltage and the second offset voltage using the voltage potential.

In some embodiments, the techniques described herein relate to a system, wherein the bias compensation circuit includes an offset capacitor, and wherein the offset capacitor stores the voltage potential indicative of the first offset voltage and the second offset voltage during a first operation mode.

In some embodiments, the techniques described herein relate to a system, wherein the offset capacitor compensates the first offset voltage and the second offset voltage during a second operation mode, and wherein the amplifier measures the electrical response in the nanopore during the second operation mode.

In some embodiments, the techniques described herein relate to a system, wherein the plurality of sequencing cells form a two-dimensional (2D) array.

In some embodiments, the techniques described herein relate to a system, wherein a sequencing cell density of the 2D array is at least 500 sequencing cells per mm2.

In some embodiments, the techniques described herein relate to a system, wherein the 2D array includes at least 1000 sequencing cells.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates an example of DNA translocation through a solid-state nanopore.

FIG. 1B schematically illustrates an example of DNA translocation through a protein nanopore.

FIG. 2A illustrates a cross-sectional view of a nanopore device according to another embodiment of the disclosed technology.

FIG. 2B illustrates an equivalent circuit model of a sequencing unit cell according to one embodiment of the disclosed technology.

FIGS. 3A and 3B depict illustrative operations performed by a first bias compensation circuit for compensating bias offsets in the common cis electrode and a trans electrode of FIG. 2B in accordance with some embodiments of the disclosed technology.

FIGS. 4A and 4B depict illustrative operations performed by a second bias compensation circuit for compensating input offset voltage of an active circuitry of FIG. 2A according to some embodiments of the disclosed technology.

FIGS. 5A and 5B depict illustrative operations performed by a third bias compensation circuit for compensating bias offsets in the common cis electrode and trans electrode of FIG. 2B and input offset voltage of an active circuitry of FIG. 2A according to some embodiments of the disclosed technology.

FIG. 6 depicts a method for compensating bias offsets in a common cis electrode and a trans electrode according to some embodiments of the disclosed technology.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a fluidic cavity) which can contain the same compositions or different compositions therein. The permeability of the membrane to any given species depends upon the nature of the membrane. In some examples, the membrane may be non-permeable to ions, to electric current, and/or to fluids. For example, a membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material. The membrane may be formed of materials of non-biological or biological origin.

An example membrane that is made from non-biological materials are block copolymer. The term is a “block copolymer” is intended to refer to a polymer having at least a first portion or block that includes a first type of monomer, and at least a second portion or bloc” that is coupled directly or indirectly to the first portion and includes a second, different type of monomer. Block copolymers include, but are not limited to, diblock copolymers and triblock copolymers. A “diblock copolymer” is intended to refer to a block copolymer that includes a first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block. A “triblock copolymer” is intended to refer to a block copolymer that includes a first, second, and third blocks coupled directly or indirectly to one another. The first and third blocks may include, or may consist essentially of, the same type of monomer (repeating unit) as one another, and the second block may include a different type of monomer (repeating unit). In one example, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block. The block copolymers may be formed into a bilayer membrane in which the hydrophilic blocks are position on the outward of the bilayer membrane and in which the hydrophobic blocks are positioned inward of the bilayer membrane.

Example hydrophilic A blocks include, but are not limited to, a polymer selected from the group consisting of: N-vinyl pyrrolidone, polyacrylamide, zwitterionic polymer (Zwitt), hydrophilic polypeptide, poly(ethylene glycol) (PEG), carbon-oxygen-nitrogen containing polymers (CₓO_{y}N_{z}), and combinations thereof. Example hydrophobic B blocks include, but are not limited to, poly(dimethylsiloxane) (PDMS), poly(isobutylene) (PiB), polybutadiene (PBd), polyisoprene, polymyrcene, polychloroprene, hydrogenated polydiene, fluorinated polyethylene, polypeptide, and combination thereof. Example block copolymers used to form a bilayer membrane include, but are not limited to, PDMS-ab-Zwitt, PiB-ab-Zwitt, PiB-ab-PEG, PiB-ab-(CₓO_{y}N_{z}), PDMS-ab-PEG, PDMS-ab-(CₓO_{y}N_{z}), PiB-aba-Zwitt, PiB-aba-PEG, PiB-aba-(CₓO_{y}N_{z}), and PDMS-aba-PEG, PDMS-ab-(CₓO_{y}N_{z}), and other suitable block copolymers,

A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

An example membrane that is made from non-biological materials are solid-state materials. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, aerolysin, CsgG, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the nanopore constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, the term “nanopore sequencer” refers to any of the devices disclosed herein that can be used for nanopore sequencing. In the examples disclosed herein, during nanopore sequencing, the nanopore is immersed in examples of the electrolyte disclosed herein and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well. Apparatuses and methods include sequencing polynucleotides and sequencing polypeptides and include providing genomics analysis and proteomics analysis.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides can be methylated bases and analysis thereof, including detecting methylated bases or detecting converted bases. These nucleotides are natural nucleotides, but it is to be further understood that non-natural nucleotides, modified nucleotides or analogs of the aforementioned nucleotides can also be used.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “bias offset voltage” and “input offset voltage” are intended to mean bias voltage difference between input terminals of an active circuitry (e.g., an amplifier) due to semiconductor process variation and/or non-ideality effect. For example, both “bias offset voltage” and “input offset voltage” of an amplifier refer to the voltage potential difference between input terminals of the amplifier. Specifically, if the amplifier is a differential amplifier, both “bias offset voltage” and “input offset voltage” refer to the voltage potential difference between two input terminals of the differential amplifier.

As used herein, the term “active circuitry” and “amplifier” may be used interchangeably to refer to semiconductor circuits with transistors that can be used to measure electrical responses of the nanopores.

The aspects and examples set forth herein and recited in the claims can be understood in view of the above definitions.

### Sequencing Using a Nanopore

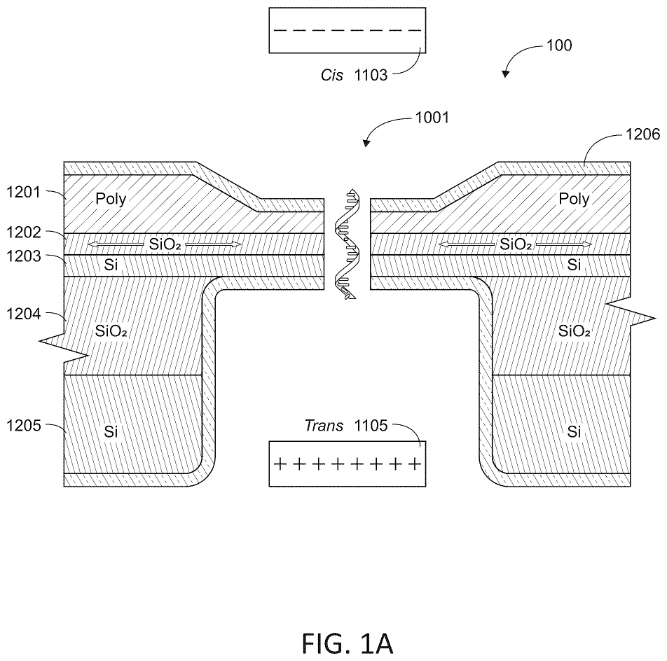

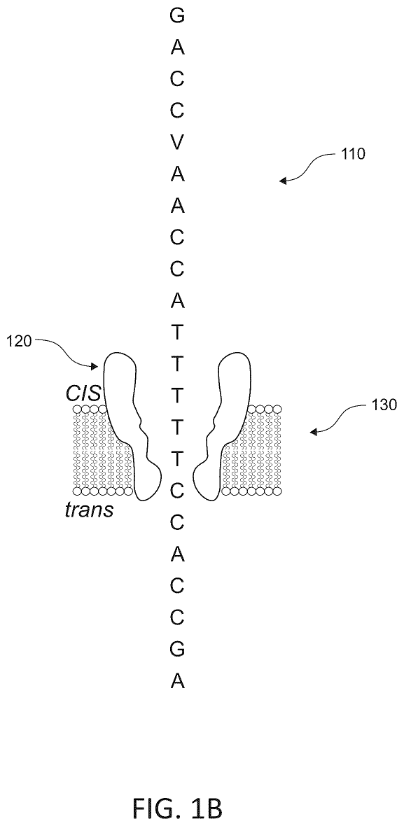

Polynucleotides may be sequenced using a nanopore unit cell, or a nanopore sensor, based on electrical responses. In some embodiments, such unit cell may include a nanopore, a flow chamber containing a liquid, one or more electrodes, and an electronic circuit (e.g., an amplifier) for measurement. In some cases, the nanopore may be a solid-state nanopore as illustrated in FIG. 1A. In some cases, the nanopore may be a solid-state nanopore directly formed as a nanoscale opening in a membrane (e.g., silicon based, graphene, or polymer membrane). A polynucleotide may be dissolved in the liquid, e.g., an electrolyte. In some embodiments, application of a voltage via the one or more electrodes results in a driving force and/or a change in the electrical conditions that are suitable for driving translocation of the polynucleotide through the nanopore, for example from the “cis” side to the “trans” side, or vice versa. As the polynucleotide translocates through the nanopore, the polynucleotide may modulate the electrical properties of the nanopore, such that the nucleobase sequence of the polynucleotide can be identified. For example, the electrical current through the nanopore or the electrical resistance at the nanopore may be a function of the identity of the nucleobase of the polynucleotide at or near the nanopore. FIG. 1A schematically illustrates an example of a polynucleotide 1001 translocating through a solid-state nanopore device 100. The solid-state nanopore device 100 includes a silicon substrate 1205; a silicon dioxide layer 1204 formed on the silicon substrate 1205; and a stack of polysilicon 1201, silicon dioxide 1202 and silicon 1203 materials formed on the silicon dioxide layer 1204. A silicon oxide layer 1206 may be grown on the surfaces of the solid-state nanopore device 100 and may insulate the solid-state nanopore device 100. A nano-scale opening is formed in the stack of polysilicon 1201, silicon dioxide 1202 and silicon 1203 materials, allowing the polynucleotide 1001 to pass through. The solid-state nanopore device 100 may further include a cis electrode 1103 and a trans electrode 1105 for application of a voltage across the solid-state nanopore device 100. An electrolyte may be filled in the chambers between the cis electrode 1103 and the trans electrode 1105 and the silicon oxide layer 1206. The polynucleotide 1001 may be negatively charged in the electrolyte and may thus be driven through the nano-scale opening from the cis side to the trans side or vice versa when a voltage difference between the cis electrode 1103 and the trans electrode 1105 is applied.

In some cases, the nanopore may be a biological nanopore formed of peptides or polynucleotides and deposited in a lipid bilayer or a polymer membrane, e.g., a synthetic polymeric membrane. In an example shown in FIG. 1B, a protein nanopore 120 is deposited in a bilayer 130, such as a block co-polymer bilayer. A single-stranded DNA 110 is passing, from the “cis” side, through the nanopore 120, to the “trans” side, or vice versa. Applying a voltage across the “cis” side to the “trans” side results in an ionic current through the nanopore. When a nucleotide of the DNA 110 is in or near a sensing zone of the nanopore, it may result in a unique ionic current blockade at the nanopore 120, and therefore a unique nanopore resistance depending on the identity of the nucleotide. By measuring the ionic current or the nanopore resistance, the nucleotide at or near the nanopore can be identified.

In other embodiments, the DNA 110 may not pass through the nanopore 120. A unique tag or label for a nucleotide in the DNA 110 may pass through the nanopore 120. In one example, a tag or label of the nucleotide may be a particular sequence combination of nucleotides. When the tag or label is in or near the nanopore, it may result in a unique ionic current blockade at the nanopore, and therefore a unique nanopore resistance depending on the identity of the molecule of interest. By measuring the ionic current or the nanopore resistance, the tag or label at or near the nanopore, and therefore the corresponding nucleotide, can be identified.

Although embodiments herein describe determining a signal level by determining the ionic current through the nanopore, embodiments also include alone or in combination determining the signal level by measuring other electrical characteristics of the cis/trans nanopore cell. For example, in other embodiments, a signal level is determined by the voltage potential at a specified area or component of the cis/trans nanopore cell. For example, in other embodiments, a signal level is determined by the electrical impedance at a specified area or component of the cis/trans nanopore cell. For example, in other embodiments, a signal level is determined by the conductivity/resistance of the nanopore membrane.

In other embodiments, sequencing of a target polynucleotide may involve nanopore sensing of (1) a single-stranded portion of the target polynucleotide; (2) a nucleic acid duplex of a portion of the target polynucleotide; (3) a label or tag that can be tethered or untethered to the target polynucleotide; or any combination thereof.

In some embodiments, multiple such nanopore unit cells may be arranged in an array, and each unit cell or each nanopore sensor may be individually accessed by a logic circuit.

### Measurement Circuit for Nanopore Sequencing

In some embodiments, a nanopore array is formed of an array of biochemical sensors, e.g., an array of nanopore unit cells described above. In some embodiments, a nanopore array can be used to perform long read DNA sequencing. A characteristic feature of a nanopore array is G-base per second per square centimeter of a chip. In some embodiments, to achieve higher data rate, the density of nanopores in a 2D array is increased. In some embodiments, a 2D readout circuit is used to take measurements from a 2D nanopore array.

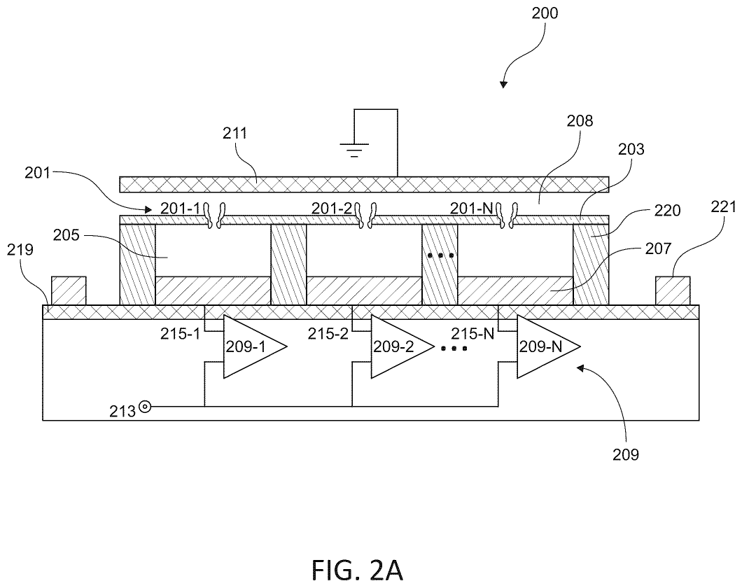

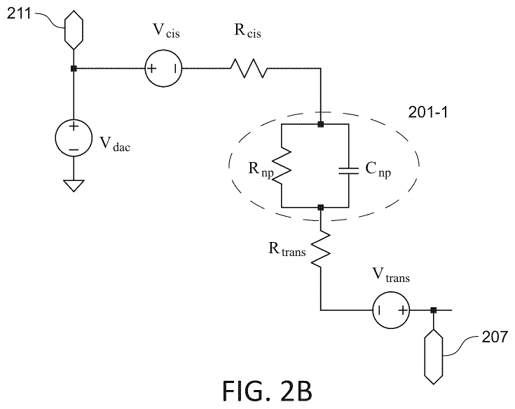

FIG. 2A schematically illustrates an embodiment of a device 200 including an amplifier array 209 integrated with a nanopore array 201. The nanopore array 201 includes the nanopore sensors 201-1, 201-2 through 201-N and the amplifier array 209 includes the amplifier 209-1, 209-2 through 209-N, with N being any positive integer number. The nanopore sensors 201-1, 201-2 through 201-N may correspond to the nanopore shown and described in conjunction with FIG. 1A or FIG. 1B. As shown in FIG. 2A, the nanopore array 201 are inserted through the membrane 203 and the nanopore array 201 are electrically connected with the amplifier array 209 through the electrolyte in the trans chamber 205 and the trans electrodes 207. Each of the nanopore sensors 201-1, 201-2 through 201-N may be electrically connected to a common cis electrode 211 through the electrolyte in the cis chamber 208. In some embodiments, the common cis electrode 211 may be the cis electrode 1103 as shown in FIG. 1A. In some embodiments, the amplifier array 209 may be fabricated as part of an application specific integrated circuit (ASIC) using complementary metal-oxide semiconductor (CMOS) process. A top surface of the ASIC may be deposited with a passivation layer 219. In some examples, the nanopore array 201 may be a two-dimensional (2D) high density nanopore array. In some embodiments, a nanopore sensor (e.g., the nanopore sensor 201-1), a corresponding trans electrode 207, and a cis electrode form a sequencing cell (or unit cell) for identifying a polynucleotide passed through the nanopore sensor; and the sequencing cell can be associated with one of the amplifiers in the amplifier array 209. The sequencing cells are separated by dielectric 220. Additionally, the device 200 may further include a ground 221 distributed on the passivation layer 219.

As illustrated in FIG. 2A, the nanopore sensor 201-1 is paired with the amplifier 209-1, the nanopore sensor 201-2 is paired with the amplifier 209-2, and the nanopore sensor 201-N is paired with the amplifier 209-N. In other words, there are N pairs of an amplifier and a nanopore sensor, with N being any positive integer number. As such, the amplifier 209-1 can be used to measure the electrical currents flowing through the nanopore sensor 201-1 for decoding the nucleic acid sequences passed through the nanopore sensor 201-1; the amplifier 209-2 can be used to measure the electrical currents flowing through the nanopore sensor 201-2 for decoding the nucleic acid sequences passed through the nanopore sensor 201-2; and the amplifier 209-N can be used to measure the electrical currents flowing through the nanopore sensor 201-N for decoding the nucleic acid sequences passed through the nanopore sensor 201-N.

As illustrated in FIG. 2A, each amplifier of the amplifier array 209 receives two inputs. More specifically, the amplifier 209-1 receives two inputs through a first input terminal 215-1 and the common input terminal (e.g., a second input terminal) 213; the amplifier 209-2 receives two inputs through a first input terminal 215-2 and the common input terminal 213; and the amplifier 209-N receives two inputs through a first input terminal 215-N and the common input terminal 213. As such, the common input terminal 213 is connected to each of the amplifier 209-1, amplifier 209-2 through amplifier 209-N. Note that, in other embodiments, the amplifier 209-1, amplifier 209-2 through amplifier 209-N may not share a common input terminal 213. In some embodiments, the amplifier 209-1 measures the electrical current flowing through the nanopore sensor 201-1 by amplifying the difference between the voltages between the first input terminal 215-1 and the common input terminal (e.g., a second input terminal) 213.

For example, the amplifier 209-1 may measure the electrical response associated with nanopore 201-1 based on voltage difference between the first input terminal 215-1 and the second input terminal 213 of the amplifier 209-1; the amplifier 209-2 may measure the electrical response associated with nanopore 201-2 based on voltage difference between the first input terminal 215-2 and the second input terminal 213 of the amplifier 209-2; and the amplifier 209-N may measure the electrical response associated with nanopore 201-N based on voltage difference between the first input terminal 215-N and the second input terminal 213 of the amplifier 209-N.

As such, there may be a plurality of unit cells for polynucleotides sequencing, where each unit cell may include a nanopore (e.g., one of the nanopores 201-1, 201-2 through 201-N), a trans chamber 205, a trans electrode 207, and an active circuitry (e.g., one of the active circuitry 209-1, 209-2 through 209-N). Each unit cell may share the cis chamber 208 that may contain, may connect to, or may be defined by a common cis electrode, such as the common cis electrode 211 shown in FIG. 2A.

By way of example for illustrating operation of a unit cell, when a polynucleotide traverses through a nanopore 201-1, the ionic current through the nanopore 201-1 may change based on the identity of the polynucleotide and the active circuitry 209-1 (e.g., an amplifier or a differential amplifier) may measure the ionic current change to identify the polynucleotide. More specifically, a nanopore 201-1 (or any other nanopore 201-2 through 201-N) may be modeled as having an equivalent resistance, i.e., the nanopore resistance Rₙₚ. The active circuitry 209-1 (or any other active circuitry 209-2 through 209-N) may then sense the voltage across the nanopore resistance Rₙₚ to identify the nucleotide that is passing through the nanopore 201-1 (or any other nanopore 201-2 through 201-N).

However, during some nanopore sequencing processes, the common cis electrode, the trans electrodes and/or the electrolyte are consumed by electrochemical processes such as redox reactions (faradaic or non-faradaic) at one or both electrodes. These electrochemical processes may result in extra voltage potentials at the common cis electrode and the trans electrodes. The extra voltage potentials may be described by the Nernst equation. These extra voltage potentials (the “offset potentials”) may affect the accuracy of the voltage bias that is intended to be applied across the nanopores 201-1, 201-2 through 201-N. In some cases, the consumptions of the common cis electrode and the trans electrodes are continuous such that the offset potentials vary at different times. Moreover, in some cases, different unit cells for polynucleotide sequencing consume the electrolyte and/or electrode(s) differently, which results in cell-to-cell variations and/or the time variation of the “offset potential.” As such, an equivalent circuit model of a unit cell that includes at least a nanopore (e.g., one of the nanopores 201-1, 201-2 through 201-N), a corresponding trans chamber 205 and trans electrode 207 and a shared cis chamber 208 that is in part defined by a common cis electrode 211 can be illustrated by the schematic as shown in FIG. 2B.

As shown in FIG. 2B, a nanopore 201-1 is modeled as a RC circuit, having a nanopore resistance Rₙₚ in parallel with a nanopore capacitance Cₙₚ. In some embodiments, the nanopore capacitance Cₙₚ may be negligible for the purpose of circuit analysis and the nanopore 201-1 may be modeled as the nanopore resistance Rₙₚ alone. In addition to modeling the nanopore 201-1 as the nanopore resistance Rₙₚ, the nanopore 201-1 may be modeled using other electrical circuit components such as capacitance or any combinations and configurations of capacitance and resistance. For example, the nanopore 201-1 may be modeled as a nanopore capacitance Cₙₚ. As another example, the nanopore 201-1 may be modeled as a nanopore resistance Rₙₚ in parallel with a nanopore capacitance Cₙₚ. As still another example, the nanopore 201-1 may be modeled as a nanopore capacitance Cₙₚ in series with a nanopore resistance Rₙₚ. It should be noted that the modeling of the nanopore 201-1 may depend on a type (e.g., a solid-state nanopore, other non-biological nanopores, a protein nanopore, and other biological nanopores) or a structure (e.g., a tunnel and a coil) of the nanopore 201-1. In some embodiments, based on the type or the structure of the nanopore 201-1, the amplifier array 209 may be configured to measure ionic current through the nanopore 201-1, the nanopore resistance Rₙₚ of the nanopore 201-1, the nanopore capacitance Cₙₚ of the nanopore 201-1, or measure any combinations of nanopore resistance Rₙₚ and nanopore capacitance Cₙₚ. It should be noted that, in addition to measuring nanopore resistance Rₙₚ, the amplifier array 209 may be configured to measure an impedance (e.g., including a magnitude and a phase) of each nanopores.

Because of the electrochemical processes described above, in addition to being set at a bias voltage V_{dac} (e.g., a constant bias voltage), the common cis electrode 211 is further modeled by a first equivalent bias V_{cis} and a first equivalent resistance R_{cis}. The first equivalent bias V_{cis} may have a time-varying or a non-constant bias voltage. In some embodiments, the first equivalent resistance R_{cis} may be negligible for the purpose of circuit analysis and the common cis electrode 211 may be modeled by the bias voltage V_{dac} and the first equivalent bias V_{cis}. As shown in FIG. 2B, the trans electrode 207 is modeled by a second equivalent bias Vₜᵣₐₙₛ and a second equivalent resistance Rₜᵣₐₙₛ. The second equivalent bias Vₜᵣₐₙₛ may have a time-varying or a non-constant bias voltage because the trans electrode 207 may be consumed by electrochemical processes (e.g., the redox reactions) during the polynucleotide sequencing process. In some embodiments, the second equivalent resistance Rₜᵣₐₙₛ may be negligible for the purpose of circuit analysis and the trans electrode 207 may be modeled by the second equivalent bias Vₜᵣₐₙₛ alone.

In view of the equivalent circuit model illustrated in FIG. 2B, an active circuitry 209-1, 209-2 or 209-N may not accurately identify the nucleotide that is passing through the nanopore 201-1, 201-2, or 201-N by measuring the voltage variation across the nanopore resistance Rₙₚ due to the presence of the first equivalent bias V_{cis} and the second equivalent bias Vₜᵣₐₙₛ. Further, because the first equivalent bias V_{cis} and the second equivalent bias Vₜᵣₐₙₛ may both be time-varying, compensating for these extra voltages may have to be performed multiple times such that voltage variation across the nanopore 201-1, 201-2 or 201-N may be correctly measured by the active circuitry 209-1, 209-2 or 209-N of a polynucleotide sequencing unit cell. The disclosed apparatus and method address this pixel-to-pixel variation and/or the time variation of the “offset potential” by providing a capacitor to store the offset potential in the electrical circuit of a pixel during the calibration phase. Then, the disclosed method applies this stored capacitor voltage to cancel out the offset potential in the electrical circuit of the pixel during the measurement phase.

In some embodiments, the disclosed apparatus and method further address input offset voltage (or bias offset voltage) between input terminals of the active circuitry 209-1, 209-2 through 209-N. Specifically, the disclosed apparatus and method further stores the bias offset voltage of the active circuitry 209-1, 209-2 through 209-N during the calibration phase and compensates the bias offset voltage of the active circuitry 209-1, 209-2 through 209-N during the measurement phase.

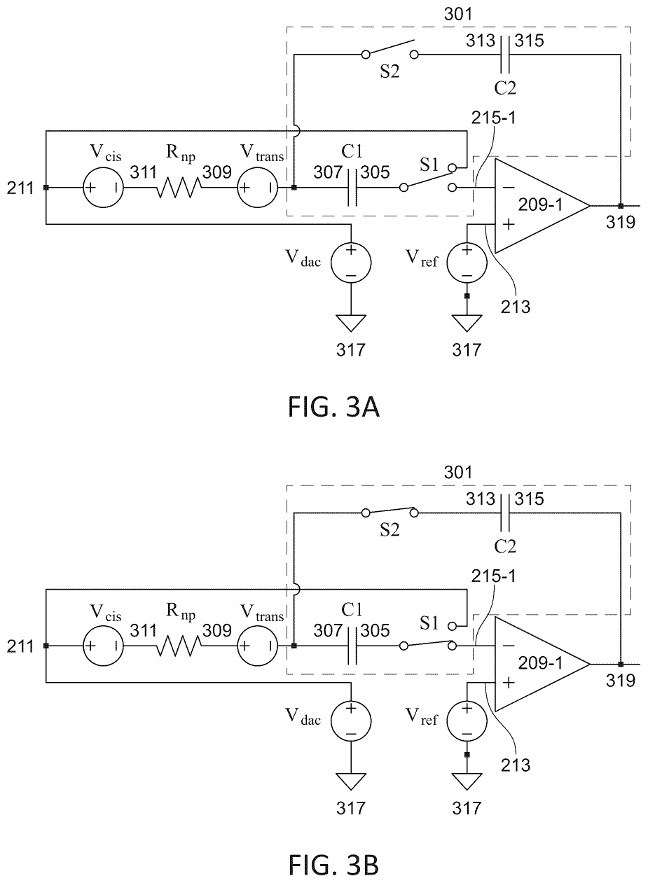

In some embodiments, the disclosed apparatus and method use auto-zero circuits to cancel out the offset potential. FIGS. 3A and 3B depict illustrative operations performed by a bias compensation circuit 301 for compensating bias offsets associated with a common cis electrode 211 and a trans electrode 207 of FIG. 2B in accordance with some embodiments of the disclosed technology. In some examples, the offset potential V_{cis}, the offset potential Vₜᵣₐₙₛ, the bias voltage V_{dac}, and the nanopore resistance Rₙₚ in FIGS. 3A and 3B may be the offset potential V_{cis}, the offset potential Vₜᵣₐₙₛ, the bias voltage V_{dac}, and the nanopore resistance Rₙₚ illustrated in FIG. 2B. Additionally, the amplifier 209-1 of FIGS. 3A and 3B may be one of the active circuitry 209-1, 209-2 through 209-N illustrated in FIG. 2A. As described above, the offset potential V_{cis} and the offset potential Vₜᵣₐₙₛ may be time-varying potentials, the bias voltage V_{dac} may be a constant voltage, and the amplifier 209-1 may sense the voltage variation across the nanopore resistance Rₙₚ during nucleotide sequencing processes. The amplifier 209-1 has a first input terminal 215-1 and a second input terminal 213. The second input terminal 213 of the amplifier 209-1 is biased at a voltage V_{ref}, which may be a constant voltage.

The bias compensation circuit 301 illustratively includes an offset capacitor C1, an integration capacitor C2, a first switch S1 and a second switch S2. The first switch S1 selectively connects a second terminal 305 of the offset capacitor C1 to the common cis electrode 211 or the first input terminal 215-1 of the amplifier 209-1. The second switch S2 selectively connects the first terminal 313 of the integration capacitor C2 to the first terminal 307 of the offset capacitor C1.

FIG. 3A shows that the bias compensation circuit 301 operates in a calibration mode, where the first switch S1 connects a second terminal 305 of the offset capacitor C1 to the common cis electrode 211 that is biased at voltage V_{dac}. During the calibration mode, the second switch S2 is open such that a first terminal 307 of the offset capacitor C1 is not connected to a first terminal 313 of the integration capacitor C2. As shown in FIG. 3A, the offset potential V_{cis}, the nanopore resistance Rₙₚ, the offset potential Vₜᵣₐₙₛ, and the offset capacitor C1 form an electrical loop (e.g., a Kirchoff's loop) that may be approximately described by the Kirchoff's voltage law, which provides that the sum of the voltage potentials around a closed loop is zero. As such, this electrical loop may be mathematically described as: V_{c1}+V_{cis}+Vₜᵣₐₙₛ=0, where V_{c1} is the voltage potential stored by the offset capacitor C1. In other words, the offset capacitor C1 inversely stores the sum of the offset potential V_{cis} and the offset potential Vₜᵣₐₙₛ. The above approximation may hold true so long as the nanopore capacitance Cₙₚ depicted in FIG. 2B is trivial enough such that voltage potential across the nanopore resistance Rₙₚ is negligible compared with the offset potential V_{cis} and the offset potential Vₜᵣₐₙₛ.

Turning now to FIG. 3B, which shows the bias compensation circuit 301 operates in a different configuration (e.g., a measurement mode) compared with FIG. 3A. As shown in FIG. 3B, the first switch S1 connects the second terminal 305 of the offset capacitor C1 to the first input terminal 215-1 of the amplifier 209-1. The second switch S2 is close such that the first terminal 307 of the offset capacitor C1 is connected to the first terminal 313 of the integration capacitor C2. Assuming the amplifier 209-1 has zero input offset voltage or the input offset voltage of the amplifier 209-1 is negligible (i.e., the voltage potential at the first input terminal 215-1 and the voltage potential at the second input terminal 213 is approximately the same), then the voltage bias V_{ref}, the offset capacitor C1, the offset potential Vₜᵣₐₙₛ, the nanopore resistance Rₙₚ, the offset potential V_{cis}, and the bias voltage V_{dac} may form a Kirchoff's loop, which can be mathematically described by: V_{ref}+V_{c1}+Vₜᵣₐₙₛ+Vₙₚ+V_{cis}+V_{dac}=0, where Vₙₚ represents the voltage potential across the nanopore resistance Rₙₚ (e.g., voltage difference between the second side 311 of the nanopore resistance Rₙₚ and the first side 309 of the nanopore resistance Rₙₚ) and V_{c1} (e.g., the voltage difference between the first terminal 307 of the offset capacitor C1 and the second terminal 305 of the offset capacitor C1) represents voltage potential stored by the offset capacitor C1 during the calibration mode. Substituting V_{c1}=−V_{cis}−Vₜᵣₐₙₛ based on the voltage potential stored by the offset capacitor C1 during the calibration mode, the Kirchoff's loop of FIG. 3B may be mathematically described as V_{ref}+Vₙₚ+V_{dac}=0. In other words, in the measurement mode, the voltage potential Vₙₚ across the nanopore resistance Rₙₚ equals −V_{ref}−V_{dac}. As such, the voltage across the nanopore resistance Rₙₚ may be independent of the offset potential V_{cis} and Vₜᵣₐₙₛ. Thus, the amplifier 209-1 may be able to more accurately measure the voltage across the nanopore resistance Rₙₚ without the extra voltages introduced due to the electrochemical processes that consume the common cis electrode 211 and the trans electrode 207 as illustrated in FIG. 2B. Advantageously, with the operation of the bias compensation circuit 301, the identity of the nucleotides that pass through a nanopore (e.g., the nanopore 201-1, 201-2 or 201-N in FIG. 2A) may be more accurately determined by measuring the voltage variation across the nanopore.

In some embodiments, the value of the offset capacitor C1 is selected to be larger than the nanopore capacitance Cₙₚ illustrated in FIG. 2B to achieve effective offset potential cancellation.

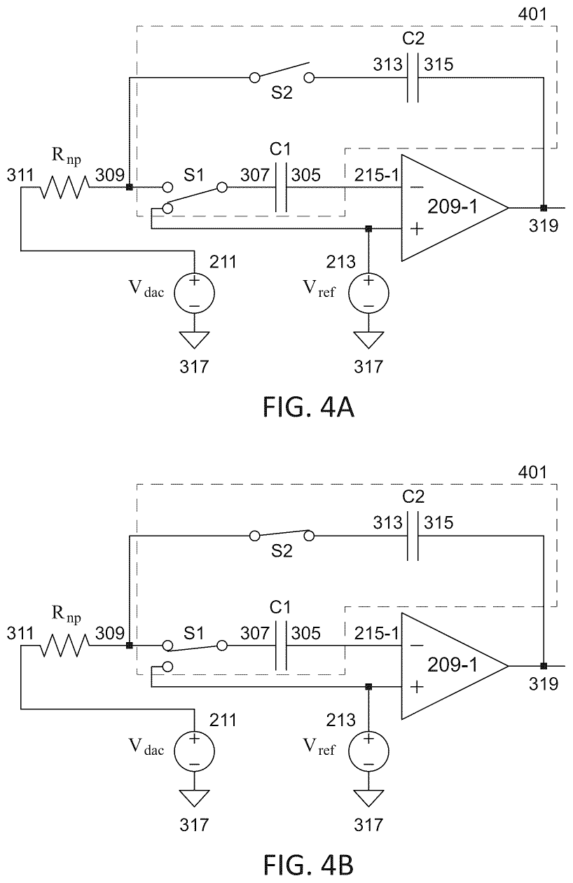

FIGS. 4A and 4B depict illustrative operations performed by a bias compensation circuit 401 for compensating input offset voltage of an amplifier (e.g., 209-1, 209-2 or 209-N) of FIG. 2A according to some embodiments of the disclosed technology. In some embodiments, the amplifier 209-1 of FIGS. 4A and 4B may be the amplifier 209-1 of FIG. 2A and the nanopore resistance Rₙₚ of FIGS. 4A and 4B may represent the equivalent circuit of the nanopore 201-1 of FIG. 2A. In some embodiments, the amplifier 209-1 and the nanopore resistance Rₙₚ as shown in FIGS. 4A and 4B may correspond to other amplifier and nanopore pairs (e.g., 209-2 and 201-2, or 209-N and 201-N) in other unit cells for polynucleotide sequencing as shown in FIG. 2A. As shown in FIGS. 4A and 4B, the bias compensation circuit 401 is coupled to the amplifier 209-1 and a nanopore (represented by the nanopore resistance Rₙₚ). The amplifier 209-1 may be a differential amplifier that senses the voltage difference/variation between the second input terminal 213 and the first input terminal 215-1 and magnifies that voltage difference/variation. In some applications, the amplifier 209-1 has to be able to sense small (e.g. in the order of mV) voltage variations in the second input terminal 213 and/or the first input terminal 215-1.

The bias compensation circuit 401 illustratively includes an offset capacitor C1, an integration capacitor C2, a first switch S1 and a second switch S2. The first switch S1 selectively connect the first terminal 307 of the offset capacitor C1 to the second input terminal 213 of the amplifier 209-1 or to the first side 309 of the nanopore resistance Rₙₚ. The second switch selectively connects the first terminal 313 of the integration capacitor C2 to the first side 309 of the nanopore resistance Rₙₚ. The second terminal 315 of the integration capacitor C2 is connected with the output 319 of the amplifier 209-1.

The bias compensation circuit 401 may utilize a calibration mode and a measurement mode to cancel out the input offset voltage of the amplifier 209-1. In some embodiments, during the calibration mode, the first switch S1 connects the first terminal 307 of the offset capacitor C1 to the second input terminal 213 of the amplifier 209-1. Additionally, the second switch S2 is open such that the first terminal 313 of the integration capacitor C2 is not connected with the first side 309 of the nanopore resistance Rₙₚ. In some embodiments, during the measurement mode, the first switch S1 connects the first terminal 307 of the offset capacitor C1 to the first side 309 of the nanopore resistance Rₙₚ. Additionally, the second switch S2 may be close such that the first terminal 307 of the offset capacitor C1, the first side 309 of the nanopore resistance Rₙₚ and the first terminal 313 of the integration capacitor C2 are connected together (e.g., having the same voltage potential) during the measurement mode.

FIG. 4A shows an example circuit configuration when the bias compensation circuit 401 operates in a calibration mode according to some embodiments of the disclosed technology. As described above, the example circuit configuration of FIG. 4A can be obtained by connecting the first terminal 307 of the offset capacitor C1 to the second input terminal 213 of the amplifier 209-1 through the first switch S1, and opening the second switch S2.

As shown in FIG. 4A, the first side 309 of the nanopore resistance Rₙₚ is neither connected to the first terminal 307 of the offset capacitor C1 nor connected to the first terminal 313 of the integration capacitor C2 because of the switching configurations of the first switch S1 and the second switch S2. The second side 311 of the nanopore resistance Rₙₚ is connected to the common cis electrode 211, which is biased at the voltage V_{dac}. The voltage at the second input terminal 213 of the amplifier 209-1 is biased at V_{ref}. With the presence of input offset voltage of the amplifier 209-1 due to process variations, the voltage at the first input terminal 215-1 of the amplifier 209-1 becomes V_{ref}+V_{offset} (assuming input offset voltage of the amplifier 209-1 equals V_{offset}). With the first switch S1 connecting the first terminal 307 of the offset capacitor C1 to the second input terminal 213 of the amplifier 209-1, a Kirchoff's loop L1 is formed between the second input terminal 213 of the amplifier 209-1, the first input terminal 215-1 of the amplifier 209-1, the second terminal 305 of the offset capacitor C1 and the first terminal 307 of the offset capacitor C1. Based on Kirchoff's loop rule, the voltage difference between the first input terminal 215-1 of the amplifier 209-1 and the second input terminal 213 of the amplifier 209-1 plus the voltage difference between the first terminal 307 of the offset capacitor C1 and the second terminal 305 of the offset capacitor C1 equals 0. With the voltage difference between the first input terminal 215-1 of the amplifier 209-1 and the second input terminal 213 of the amplifier 209-1 being V_{offset}, the offset capacitor C1 is charged to −V_{offset} (i.e., the voltage difference between the first terminal 307 and the second terminal 305 equals −V_{offset}).

FIG. 4B shows an example circuit configuration when the bias compensation circuit 401 operates in a measurement mode according to some embodiments of the disclosed technology. As described above, the example circuit configuration of FIG. 4B can be obtained by connecting the first terminal 307 of the offset capacitor C1 to the first side 309 of the nanopore resistance Rₙₚ through the first switch S1, and closing the second switch S2.

As illustrated in FIG. 4B, the voltage at the second input terminal 213 of the amplifier 209-1 is set as V_{ref} and the voltage at the common cis electrode 211 is biased at V_{dac}. As such, the voltage at the first input terminal 215-1 of the amplifier 209-1 remains V_{ref}+V_{offset}. With the first switch S1 connecting the first terminal 307 of the offset capacitor C1 to the first side 309 of the nanopore resistance Rₙₚ, a Kirchoff's loop is formed by traversing from the ground potential 317 to the second input terminal 213 of the amplifier 209-1, the first input terminal 215-1 of the amplifier 209-1, the second terminal 305 of the offset capacitor C1, the first terminal 307 of the offset capacitor C1, the first side 309 of the nanopore resistance Rₙₚ and the second side 311 of the nanopore resistance Rₙₚ, the common cis electrode 211, and returning back to the ground potential 317. Based on the Kirchoff's loop rule, V_{ref}+V_{offset}+(−V_{offset})+Vₙₚ+(−V_{dac})=0, where Vₙₚ represents the voltage difference across the nanopore sensor (e.g., voltage difference between the second side 311 of the nanopore resistance Rₙₚ and the first side 309 of the nanopore resistance Rₙₚ). As such, the voltage across the nanopore resistance Rₙₚ equals V_{dac}−V_{ref}, which is independent of the input offset voltage of the amplifier 209-1. Advantageously, the cancellation of the input offset voltage of an amplifier (e.g., amplifier 209-1, 209-2 and/or 209-N in FIG. 2A) may lead to better measurement for nanopore sequencing operations that can be performed by the nanopore device 200 illustrated in FIG. 2A.

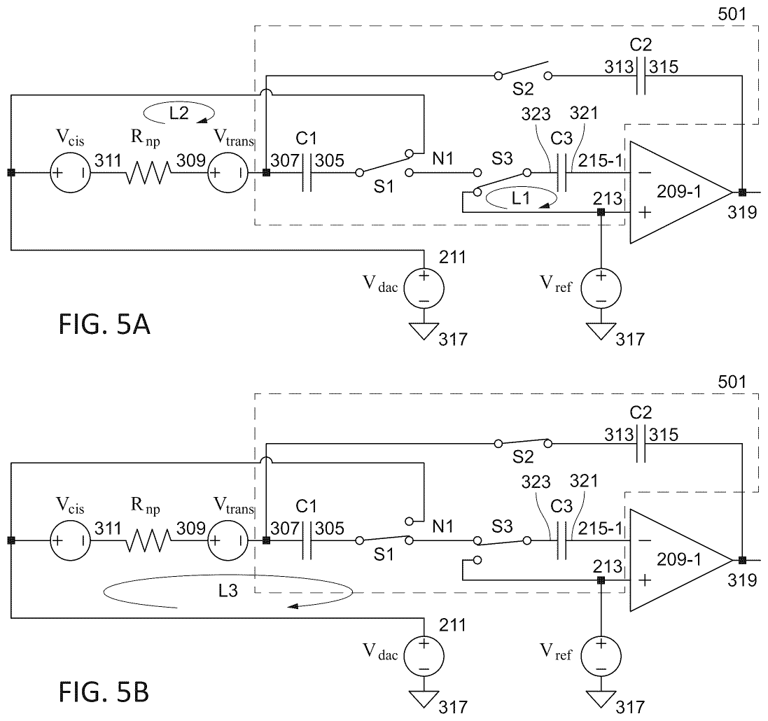

In some embodiments, the bias offsets associated with the electrode(s) and/or electrolyte may be canceled out through a combined compensation circuit. FIGS. 5A and 5B depict illustrative operations performed by a bias compensation circuit 501 for compensating bias offsets associated with a common electrode and a trans electrode (e.g., the common cis electrode 211 and trans electrode 207 of FIG. 2B) as well as for compensating input offset voltage of an amplifier (e.g., 209-1, 209-2 or 209-N of FIG. 2A) in accordance with some embodiments of the disclosed technology. In some examples, the amplifier 209-1 of FIGS. 5A and 5B may be the amplifier 209-1 of FIG. 2A and the nanopore resistance Rₙₚ of FIGS. 5A and 5B may represent the equivalent circuit of the nanopore 201-1 of FIG. 2A. In some examples, the amplifier 209-1 and the nanopore resistance Rₙₚ as shown in FIGS. 5A and 5B may correspond to other amplifier and nanopore pairs (e.g., 209-2 and 201-2, or 209-N and 201-N) in other unit cells for polynucleotide sequencing as shown in FIG. 2A. In some examples, the offset potential V_{cis}, the offset potential Vₜᵣₐₙₛ, the bias voltage V_{dac}, and the nanopore resistance Rₙₚ in FIGS. 5A and 5B may be the offset potential V_{cis}, the offset potential Vₜᵣₐₙₛ, the bias voltage V_{dac}, and the nanopore resistance Rₙₚ illustrated in FIG. 2B.

As shown in FIGS. 5A and 5B, the bias compensation circuit 501 is coupled to the amplifier 209-1 and a nanopore (represented by the nanopore resistance Rₙₚ). The amplifier 209-1 may be a differential amplifier that senses the voltage difference/variation between the second input terminal 213 and the first input terminal 215-1 and magnifies that voltage difference/variation. In some applications, the amplifier 209-1 has to be able to sense small (e.g. in the order of mV) voltage variations in the second input terminal 213 and/or the first input terminal 215-1.

The bias compensation circuit 501 illustratively includes a first offset capacitor C1, an integration capacitor C2, a second offset capacitor C3, a first switch S1, a second switch S2, a third switch S3, and a node N1. The first switch S1 selectively connects a second terminal 305 of the first offset capacitor C1 to the common cis electrode 211 or the node N1. The second switch S2 selectively connects the first terminal 313 of the integration capacitor C2 to the first terminal 307 of the first offset capacitor C1. The third switch S3 selectively connects the first terminal 323 of the second offset capacitor C3 to the second input terminal 213 of the amplifier 209-1 or the node N1. Additionally, the second terminal 315 of the integration capacitor C2 is connected to the output 319 of the amplifier 209-1.

The bias compensation circuit 501 may utilize a calibration mode and a measurement mode to cancel out the offset potential V_{cis}, the offset potential Vₜᵣₐₙₛ and the input offset voltage of the amplifier 209-1. In some embodiments, during the calibration mode, the first switch S1 connects the second terminal 305 of the offset capacitor C1 to the common cis electrode 211. Additionally, the second switch S2 is open such that the first terminal 313 of the integration capacitor C2 is not connected with the first terminal 307 of the offset capacitor C1. Further, during the calibration mode, the third switch S3 connects the first terminal 323 of the second offset capacitor C3 to the second input terminal 213 of the amplifier 209-1.

In some embodiments, during the measurement mode, the first switch S1 connects the second terminal 305 of the offset capacitor C1 to the node N1. Additionally, the third switch S3 connects the first terminal 323 of the second offset capacitor C3 to the node N1. As such, the first terminal 323 of the second offset capacitor C3 and the second terminal 305 of the first offset capacitor C1 are connected with each other. Further, the second switch S2 is close such that the first terminal 307 of the first offset capacitor C1 and the first terminal 313 of the integration capacitor C2 are connected with each other during the measurement mode.

FIG. 5A shows an example circuit configuration when the bias compensation circuit 501 operation in a calibration mode according to some embodiments of the disclosed technology. As shown in FIG. 5A, node N1 is left floating because of the switching configuration of the first switch S1 and the third switch.

The voltage at the second input terminal 213 of the amplifier 209-1 is biased at V_{ref}. The first terminal 323 of the second offset capacitor C3 is connected to the second input terminal 213 of the amplifier 209-1. With the presence of input offset voltage of the amplifier 209-1, the voltage at the first input terminal 215-1 of the amplifier 209-1 becomes V_{ref}+V_{offset} (assuming input offset voltage of the amplifier 209-1 equals V_{offset}). With the third switch S3 connecting the first terminal 323 of the second offset capacitor C3 to the second input terminal 213 of the amplifier 209-1, a Kirchoff's loop L1 is formed between the second input terminal 213 of the amplifier 209-1, the first input terminal 215-1 of the amplifier 209-1, the second terminal 321 of the second offset capacitor C3 and the first terminal 323 of the second offset capacitor C3. Based on Kirchoff's loop rule, the voltage difference between the first input terminal 215-1 of the amplifier 209-1 and the second input terminal 213 of the amplifier 209-1 plus the voltage difference between the first terminal 323 of the second offset capacitor C3 and the second terminal 321 of the second offset capacitor C3 equals 0. With the voltage difference between the first input terminal 215-1 of the amplifier 209-1 and the second input terminal 213 of the amplifier 209-1 being V_{offset}, the second offset capacitor C3 is charged to −V_{offset} (i.e., the voltage difference between the first terminal 323 and the second terminal 321 equals −V_{offset}).

Concurrently, during the calibration mode, the first switch S1 connects the second terminal 305 of the first offset capacitor C1 to the common cis electrode 211. As such a Kirchoff's loop L2 is formed between the second terminal 305 of the first offset capacitor C1, the first terminal 307 of the first offset capacitor C1, the offset potential Vₜᵣₐₙₛ, the nanopore resistance Rₙₚ, and the offset potential V_{cis}. Assuming the voltage potential across the nanopore resistance Rₙₚ is negligible compared with the offset potential Vₜᵣₐₙₛ and the offset potential V_{cis}, the Kirchoff's loop L2 can be mathematically formulated as: V_{c1}+V_{cis}+Vₜᵣₐₙₛ=0, where V_{c1} is the voltage potential stored by the offset capacitor C1. Thus, the voltage stored by C1 (i.e., the voltage difference between the first terminal 307 and the second terminal 305) during the calibration mode is −(V_{cis}+Vₜᵣₐₙₛ).

FIG. 5B shows an example circuit configuration when the bias compensation circuit 501 operates in a measurement mode according to some embodiments of the disclosed technology. As illustrated in FIG. 5B, the first switch S1 connects the second terminal 305 of the first offset capacitor C1 to the node N1, and the third switch S3 also connects the first terminal 323 of the second offset capacitor C3 to the node N1. As such, the first terminal 323 of the second offset capacitor C3 is connected with the second terminal 305 of the first offset capacitor C1. During the measurement mode, the voltage at the second input terminal 213 of the amplifier 209-1 is set as V_{ref} and the voltage at the common cis electrode 211 is biased at V_{dac}. Thus, the voltage at the first input terminal 215-1 of the amplifier 209-1 is V_{ref}+V_{offset}.

With the first terminal 323 of the second offset capacitor C3 connected with the second terminal 305 of the first offset capacitor C1, a Kirchoff's loop L3 is formed by traversing from the ground potential 317 to the second input terminal 213 of the amplifier 209-1, the first input terminal 215-1 of the amplifier 209-1, the second terminal 321 of the second offset capacitor C3, the first terminal 323 of the second offset capacitor C3, the second terminal 305 of the first offset capacitor C1, the first terminal 307 of the first offset capacitor C1, the offset potential Vₜᵣₐₙₛ, the first side 309 of the nanopore resistance Rₙₚ, the second side 311 of the nanopore resistance Rₙₚ, the offset potential V_{cis}, the common cis electrode 211, and returning back to the ground potential 317.

Based on the Kirchoff's loop rule, V_{ref}+V_{offset}+V_{c3}+V_{c1}+Vₜᵣₐₙₛ+Vₙₚ+V_{cis}+(−V_{dac})=0, where Vₙₚ represents the voltage difference across the nanopore sensor (e.g., voltage difference between the second side 311 of the nanopore resistance Rₙₚ and the first side 309 of the nanopore resistance Rₙₚ), V_{c3} represents the voltage stored by the second offset capacitor C3 during the calibration mode, and V_{c1} represents the voltage stored by the first offset capacitor C1 during the calibration mode. Substituting V_{c3} by −V_{offset} and V_{c1} by −(V_{cis}+Vₜᵣₐₙₛ), the Kirchoff's loop L3 can be mathematically reformulated as: V_{ref}+V_{offset}+(−V_{offset})−(V_{cis}+Vₜᵣₐₙₛ)+Vₜᵣₐₙₛ+Vₙₚ+V_{cis}+(−V_{dac})=0. Thus, the voltage Vₙₚ across the nanopore resistance Rₙₚ equals V_{dac}−V_{ref}, which is independent of the input offset voltage of the amplifier 209-1. Advantageously, the cancellation of the input offset voltage of an amplifier (e.g., amplifier 209-1, 209-2 and/or 209-N in FIG. 2A) and the cancellation of the offsets associated with the common cis electrode 211 and the trans electrode 207 may lead to better measurement for nanopore sequencing operations that can be performed by the nanopore device 200 illustrated in FIG. 2A.

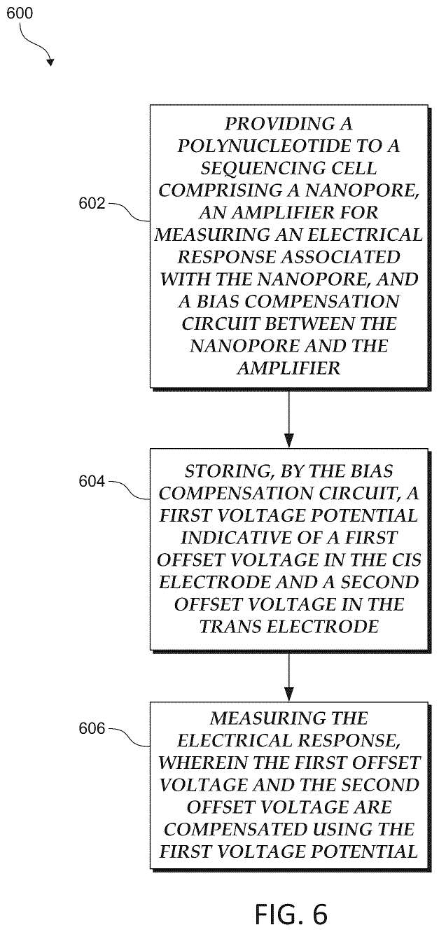

FIG. 6 is a flow diagram depicting a method 600 for compensating offset voltages in the common cis electrode 211 and a trans electrode 207 of FIG. 2A in accordance with some embodiments of the disclosed technology. Block 602 includes providing a polynucleotide to a sequencing cell that includes a nanopore, an amplifier, and a bias compensation circuit. The sequencing cell may include a nanopore, such as one of 201-1, 201-2 through 201-N shown in FIG. 2A, and a trans chamber and a trans electrode corresponding to the nanopore. In some embodiments, a plurality of sequencing cells form a nanopore array, together with a common cis electrode 211 and a cis chamber 208 to form a microwell layer of a sequencing device.

In some embodiments, the amplifier may be one of the active circuitry 209-1, 209-2 through 209-N shown in FIG. 2A. For example, the amplifier may be the active circuitry 209-1 that is configured to measure an electrical response associated with the nanopore 201-1 when the polynucleotide is around the nanopore 201-1. In some embodiments, the bias compensation circuit may be the bias compensation circuit 301 illustrated in FIGS. 3A and 3B. In some embodiments, the bias compensation circuit may be the bias compensation circuit 501 illustrated in FIGS. 5A and 5B.

At block 604, the bias compensation circuit stores a first voltage potential that is indicative of a first offset voltage in the cis electrode and a second offset voltage in the trans electrode. For example, with reference to FIGS. 3A and 5A, the first voltage potential may be stored in the first offset capacitor C1 during the calibration mode.

At block 606, the electrical response in the nanopore of the sequencing cell is measured, where the first offset voltage and the second offset voltage are compensated using the first voltage potential stored by the bias compensation circuit. Although not shown in FIG. 6, in some embodiments, an electrolyte may be provided to a sequencing cell before the polynucleotide is provided to the sequencing cell.

In some embodiments, the applied voltage value or waveform of each of the “normal operation” modes, “off” mode or “negative bias” mode can be chosen depending on the experimental conditions and requirements. In some embodiments, the applied voltage value or waveform of each of the “normal operation” modes, “off” mode or “negative bias” mode can vary over time. In some embodiments, the applied voltage value or waveform of each of the “normal operation” modes, “off” mode or “negative bias” mode can be controlled independently in each unit cell. In some examples, the normal operation modes may use positive voltage biases. For example, the positive biases may include several different values, such as 40 mV, 60 mV, 80 mV, etc.

To independently control or address each unit cell, in some embodiments, each of the nanopore unit cells in a nanopore array may have its own trans electrode but may share a common cis electrode. In some embodiments, each of the nanopore unit cells in a nanopore array may have its own cis electrode but may share a common trans electrode. In some embodiments, each of the nanopore unit cells in a nanopore array may have its own cis electrode and trans electrode. In some embodiments, each of the nanopore unit cells in a nanopore array may share a common cis electrode and a common trans electrode.

The array may have any suitable number of nanopore unit cells. In some instances, the array comprises about 200, about 400, about 600, about 800, about 1000, about 1500, about 2000, about 3000, about 4000, about 5000, about 10000, about 15000, about 20000, about 40000, about 60000, about 80000, about 100000, about 200000, about 400000, about 600000, about 800000, about 1000000, about 10000,000 or more nanopore unit cells. In some instances, the array comprises at least 200, at least 400, at least 600, at least 800, at least 1000, at least 1500, at least 2000, at least 3000, at least 4000, at least 5000, at least 10000, at least 15000, at least 20000, at least 40000, at least 60000, at least 80000, at least 100000, at least 200000, at least 400000, at least 600000, at least 800000, at least 1000000 or at least 10000000 nanopore unit cells. In some cases, the array can include individually addressable nanopore unit cells at a density of at least about 500, 600, 700, 800, 900, 1000, 10,000, 100,000 or 1,000,000 unit cells per mm².

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A device for sequencing polynucleotides comprising at least one sequencing cell, wherein the at least one sequencing cell comprises:
a nanopore disposed between a cis electrode and a trans electrode for sensing a polynucleotide;
an amplifier configured to measure an electrical response associated with the nanopore; and
a bias compensation circuit coupled to the nanopore and the amplifier, the bias compensation circuit configured to:
store a first voltage potential indicative of a first offset voltage in the cis electrode and a second offset voltage in the trans electrode; and
compensate the first offset voltage and the second offset voltage using the first voltage potential.

2. The device of claim 1, wherein the bias compensation circuit comprises a first offset capacitor, and wherein the first offset capacitor stores the first voltage potential indicative of the first offset voltage in the cis electrode and the second offset voltage in the trans electrode during a first operation mode.

3. The device of claim 2, wherein the first offset capacitor has a first terminal and a second terminal, and wherein during the first operation mode:
the first terminal is connected to the trans electrode; and
the second terminal is operably connected to the cis electrode.

4. The device of claim 2, wherein the first offset capacitor compensates the first offset voltage and the second offset voltage using the first voltage potential during a second operation mode, and wherein the amplifier measures the electrical response associated with the nanopore during the second operation mode.

5. The device of claim 4, wherein the amplifier has a bias offset voltage between a first input terminal and a second input terminal, and wherein the bias compensation circuit comprises a second offset capacitor that stores a second voltage potential indicative of the bias offset voltage during the first operation mode.

6. The device of claim 5, wherein the second offset capacitor has a third terminal and a fourth terminal, and wherein during the first operation mode:
the third terminal is operably connected to the second input terminal; and
the fourth terminal is connected to the first input terminal.

7. The device of claim 5, wherein the second offset capacitor compensates the bias offset voltage using the second voltage potential during the second operation mode.

8. The device of claim 1, wherein the first offset voltage results from a first redox reaction in the cis electrode and the second offset voltage results from a second redox reaction in the trans electrode.

9. The device of claim 1, wherein the first offset voltage and the second offset voltage are time-varying.

10. The device of claim 1, wherein the nanopore is a polypeptide nanopore or a solid- state nanopore, and wherein the polypeptide nanopore forms an opening in a lipid, polymer, or solid-state membrane.

11. The device of claim 1, wherein the electrical response is an ionic current through the nanopore, and wherein the ionic current is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

12. A method for sequencing polynucleotides, comprising:
providing a polynucleotide to a sequencing cell comprising a nanopore disposed between a cis electrode and a trans electrode, an amplifier for measuring an electrical response associated with the nanopore, and a bias compensation circuit between the nanopore and the amplifier;
storing, by the bias compensation circuit, a first voltage potential indicative of a first offset voltage in the cis electrode and a second offset voltage in the trans electrode; and
measuring the electrical response, wherein the first offset voltage and the second offset voltage are compensated using the first voltage potential.

13. The method of claim 12, wherein the first voltage potential is stored in a first offset capacitor of the bias compensation circuit during a first operation mode.

14. The method of claim 13, wherein storing the first voltage potential comprises:
connecting a first terminal of the first offset capacitor to the trans electrode; and
connecting a second terminal of the first offset capacitor to the cis electrode.

15. The method of claim 14, wherein measuring the electrical response comprises:
connecting the first terminal of the first offset capacitor to the trans electrode; and
connecting the second terminal of the first offset capacitor to a first input terminal of the amplifier.

16. The method of claim 15, wherein the amplifier has a bias offset voltage between the first input terminal and a second input terminal, the method further comprising: storing, by the bias compensation circuit, a second voltage potential indicative of the bias offset voltage during the first operation mode.

17. The method of claim 16, wherein measuring the electrical response further comprises compensating the bias offset voltage using the second voltage potential during a second operation mode.

18. The method of claim 12, wherein the electrical response measured by the amplifier is an ionic current through the nanopore or equivalents thereof.

19. The method of any of claim 12, further comprising providing an electrolyte to the sequencing cell prior to providing the polynucleotide.

20. A system for sequencing polynucleotides, comprising:
a plurality of sequencing cells, each of the plurality of sequencing cells comprising:
a nanopore disposed between a cis electrode and a trans electrode for sensing a polynucleotide;
an amplifier configured to measure an electrical response in the nanopore; and
a bias compensation circuit configured to:
store a voltage potential indicative of a first offset voltage in a common cis electrode and a second offset voltage in the trans electrode; and
compensate the first offset voltage and the second offset voltage using the voltage potential.

21. The system of claim 20, wherein the bias compensation circuit comprises an offset capacitor, and wherein the offset capacitor stores the voltage potential indicative of the first offset voltage and the second offset voltage during a first operation mode.

22. The system of claim 21, wherein the offset capacitor compensates the first offset voltage and the second offset voltage during a second operation mode, and wherein the amplifier measures the electrical response in the nanopore during the second operation mode.

23. The system of claim 20, wherein the plurality of sequencing cells form a two-dimensional (2D) array.

24. The system of claim 23, wherein a sequencing cell density of the 2D array is at least 500 sequencing cells per mm².

25. The system of claim 23, wherein the 2D array includes at least 1000 sequencing cells.
